(19) United States
(12) Patent Application Publication
(10) Pub. No.: US 2018/0305298 A1
(43) Pub. Date: Oct. 25, 2018

(54) DEUTERATED CHLOROKYNURENINES FOR THE TREATMENT OF NEUROPSYCHIATRIC DISORDERS

(71) Applicant: AUSPEX PHARMACEUTICALS, INC., La Jolla, CA (US)

(72) Inventors: Gregory R. OTT, Media, PA (US); Chengzhi ZHANG, San Diego, CA (US); Ralph LAUFER, Tel Aviv (IL)

(21) Appl. No.: 15/767,768

(22) PCT Filed: Sep. 8, 2016
(86) PCT No.: PCT/US2016/050667

§ 371 Date: Apr. 12, 2018

Related U.S. Application Data
(60) Provisional application No. 62/242,727, filed on Oct. 16, 2015.

(51) Int. Cl. C07C 229/42; C07B 59/00; A61K 31/197; A61K 45/06; A61P 25/08

(52) U.S. Cl. CPC C07C 229/42; C07B 59/001; A61P 25/08; A61K 45/06; A61K 31/197

(57) ABSTRACT

Described are deuterated chlorokynurenines and compositions, and their application as pharmaceuticals for the treatment of disease. Methods of modulating N-methyl-D-aspartate (NMDA) receptor activity, methods of treating disorders, including neuropsychiatric disorders such as depression, epilepsy, schizophrenia, and Huntington's Disease, and use of said deuterated chlorokynurenines are also described.

### CROSS-REFERENCE TO RELATED APPLICATIONS

This application claims the benefit of priority of U.S. provisional Application No. 62/242,727, filed Oct. 16, 2015, the disclosure of which is hereby incorporated by reference, as if written herein, in its entirety.

### TECHNICAL FIELD

Disclosed herein are new deuterated chlorokynurenines and compositions and their application as pharmaceuticals for the treatment of disease. Methods of modulation of N-methyl-D-aspartate (NMDA) receptor activity and of treatment of disorders, including neuropsychiatric disorders such as depression, epilepsy, schizophrenia, and Huntington's Disease in a subject are also provided.

### BACKGROUND

### 4-Chlorokynurenine ((2S)-2-Amino-4-(2-amino-4-chlorophenyl)-4-oxobutanoic acid; 4-Cl—KYN

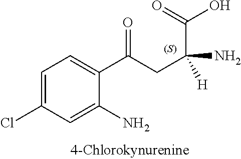

is an analogue of L-kynurenine, which is a metabolite of the amino acid tryptophan. L-kynurenine can be metabolized through three distinct pathways: the kynurenine aminotransferases (KAT) pathway, the kynureninase pathway, and the kynurenine monooxygenase (KMO) pathway.

Following the first biosynthetic pathway noted above, 4-chlorokynurenine has been demonstrated to be a prodrug of 7-chlorokynurenic acid (7-CKA) via metabolism by kynurenine aminotransferases (KATs). 7-Chlorokynurenic acid is an analogue of kynurenic acid, a natural neuroactive compound with anti-excitotoxic and anticonvulsant properties. Regulation of the conversion of 4-chlorokynurenine into active 7-chlorokynurenic acid thus has significant therapeutic importance. 7-Chlorokynurenic acid is itself a potent and selective noncompetitive agonist of the glycine B (GlyB) coagonist regulatory site on the NMDA receptor and a competitive vesicular glutamate reuptake inhibitor:

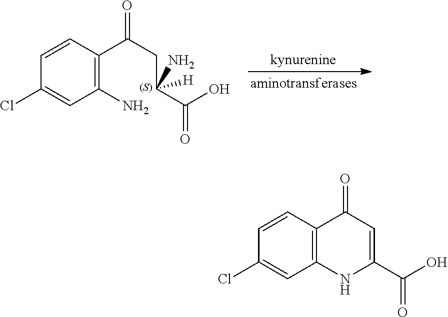

7-Chlorokynurenic acid produces rapid antidepressant effects in animal models of depression, and unlike classic NMDA receptor antagonists (such as ketamine, phencyclidine (PCP), lanicemine, and dizocilpine) which block the ion channel, 7-chlorokynurenic acid down-regulates receptor activity. Clinical studies also indicate it may be useful for the treatment of pain. However, 7-chlorokynurenic acid is unable to cross the blood-brain barrier, and for this reason, is limited in clinical utility. By contrast, 4-chlorokynurenine is efficiently and rapidly transported across the blood-brain barrier (BBB), and is converted in the brain into 7-chlorokynurenic acid. Accordingly, 4-chlorokynurenine is being studied in clinical trials as a potential treatment for major depressive disorder. Additionally, the expression of the KAT enzymes is significantly upregulated in areas of inflammation, neuronal damage, and other pathological processes, which results in a local increase in the production of 7-chlorokynurenic acid, which may result in a focal increase of active drug in the regions of pathology and greatest therapeutic need.

Following the second biosynthetic pathway noted above, 4-chlorokynurenine has been demonstrated to be a precursor in the synthesis of 4-chloro-3-hydroxyanthranilic acid by kynureninase, which catalyzes the conversion of kynurenine to 3-hydroxy anthranilic acid. 4-chloro-3-hydroxyanthranilic acid is a potent inhibitor of 3-hydroxyanthranilate oxidase (3-HAO), the enzyme responsible for the production of quinolinic acid, a potent NMDA receptor agonist, convulsant, and endogenous excitotoxic brain constituent. Abnormal increase in the quinolinic acid/kynurenic acid ratio in the brain has been associated with seizures and excitotoxic neurodegeneration, as well as neuropsychiatric pathologies such as Huntington's disease, seizures, and depression, and schizophrenia. 4-chloro-3-hydroxyanthranilic acid has also been shown to preserve white matter and effect functional recovery in a model of spinal cord injury, without enhancing kynurenic acid levels.

In a third pathway, kynurenine is converted by kynurenine monooxygenase to 3-hydroxykynurenine, and from there to downstream products including quinolinic acid.

7-Chlorokynurenic acid may also exert at least some of its effects through the α7-nicotininc acid acetylcholine receptor (α7nAChR). Dysregulation of prefrontal α7nAChRs might be central to these behavioral and chemical abnormalities. Thus, α7nAChR protein levels are reduced in the prefrontal cortex of individuals with schizophrenia, and the α7nAChR gene and a schizophrenia endophenotype (disrupted P50 evoked response to repeated auditory stimuli) are linked to the same locus and associated with disease transmission. Moreover, specific cognitive improvements in schizophrenia patients can be achieved by galantamine, probably by activating the allosteric potentiating ligand (APL) site of the α7nAChR. Notably, α7nAChRs in the mammalian brain are frequently localized presynaptically on glutamatergic nerve terminals, where they regulate the release of glutamate. Kynurenic acid levels are abnormally high in the brain and cerebrospinal fluid of schizophrenia patients, and endogenous kynurenic acid appears to function as a preferential α7nAChR antagonist.

### SUMMARY

Provided are deuterium-substituted chlorokynurenine compounds, which are N-methyl-D-aspartate (NMDA) receptor modulators. Also provided are pharmaceutical compositions comprising the deuterium-substituted chlorokynurenine compounds, and methods of use thereof, including methods for treatment or prevention of methyl-D-aspartate (NMDA) receptor-mediated disorders by administering, to a patient, the deuterium-substituted chlorokynurenine compounds or pharmaceutical compositions comprising the deuterium-substituted chlorokynurenine compounds. Further provided are methods of synthesizing the deuterium-substituted chlorokynurenine compounds.

### DETAILED DESCRIPTION

Deuterium Kinetic Isotope Effect

Metabolic reactions catalyzed in vivo by enzymes, for example those of the kynureninase and kynurenine monooxygenase metabolic pathways, frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C-¹H bond. If a C-¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C-¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching, which can occur when compounds, sequestered by enzymes, bind transiently and re-bind in a variety of conformations prior to the chemical reaction (e.g., oxidation). Metabolic switching is enabled by the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

The carbon-hydrogen bonds of 4-chlorokynurenine and 7-chlorokynurenic acid contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could affect the pharmacokinetic, pharmacologic and/or toxicologic profiles of such siponimod in comparison with the compound having naturally occurring levels of deuterium.

Deuteration of 4-chlorokynurenine thus has the potential to alter its rate of conversion into 7-chlorokynurenic acid, and to alter the rate of degradation of 7-chlorokynurenic acid itself. Limiting these rates has the potential to decrease the danger of the administration of such drugs and may even allow increased dosage and/or increased efficacy. These transformations may occur through polymorphically-expressed enzymes, exacerbating interpatient variability. Further, some disorders are best treated when the subject is medicated around the clock or for an extended period of time. For all of the foregoing reasons, a medicine with a longer half-life may result in greater efficacy and cost savings. Various deuteration patterns can be used to (a) reduce or eliminate unwanted metabolites, (b) increase the half-life of the prodrug or drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not.

Accordingly, provided herein are compounds of structural Formula I:

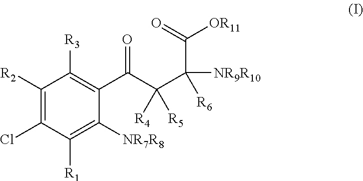

or a salt thereof, wherein:

R₁-R₁₁ are independently selected from the group consisting of hydrogen and deuterium; and

at least one of R₁-R₁₁ is deuterium.

In certain embodiments of the present invention, compounds have structural Formula Ia:

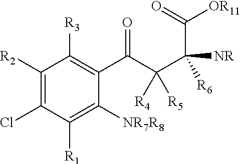

or a salt thereof, wherein:

R₁-R₁₁ are independently selected from the group consisting of hydrogen and deuterium; and

at least one of R₁-R₁₁ is deuterium.

Without intending to be bound by theory, it is understood that while compounds of Formula I and Formula Ia containing the moiety

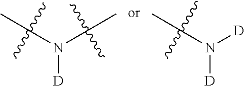

can be synthesized, it is believed that it is unlikely that the deuterium atom(s) on these moieties will be retained after the compound is administered to a subject. When administered to a subject, it is thought that the deuterium in the moiety

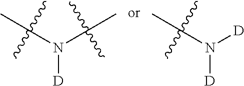

will be exchanged with a hydrogen atom to produce the moiety

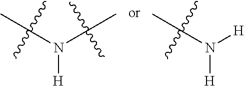

due to the large amount of water present in a subject's body. Accordingly, embodiments of the invention encompass compounds comprising one or more

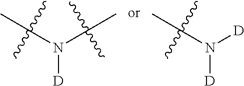

moieties in addition to one or more

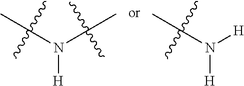

moieties.

In certain embodiments of the present invention, compounds have structural Formula II:

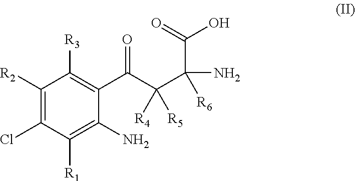

or a salt thereof, wherein:

R₁-R₆ are independently selected from the group consisting of hydrogen and deuterium; and

at least one of R₁-R₆ is deuterium.

In certain embodiments of the present invention, compounds have structural Formula IIa:

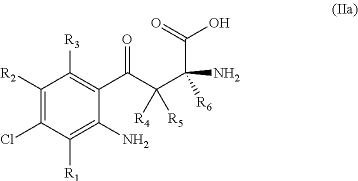

or a salt thereof, wherein:

R₁-R₆ are independently selected from the group consisting of hydrogen and deuterium; and

at least one of R₁-R₆ is deuterium.

In certain embodiments, at least one of R₁-R₆ independently has deuterium enrichment of no less than about 10%. In certain embodiments, at least one of R₁-R₆ independently has deuterium enrichment of no less than about 50%. In certain embodiments, at least one of R₁-R₆ independently has deuterium enrichment of no less than about 90%. In certain embodiments, at least one of R₁-R₆ independently has deuterium enrichment of no less than about 98%.

In certain embodiments, the compound has a structural formula selected from the group consisting of

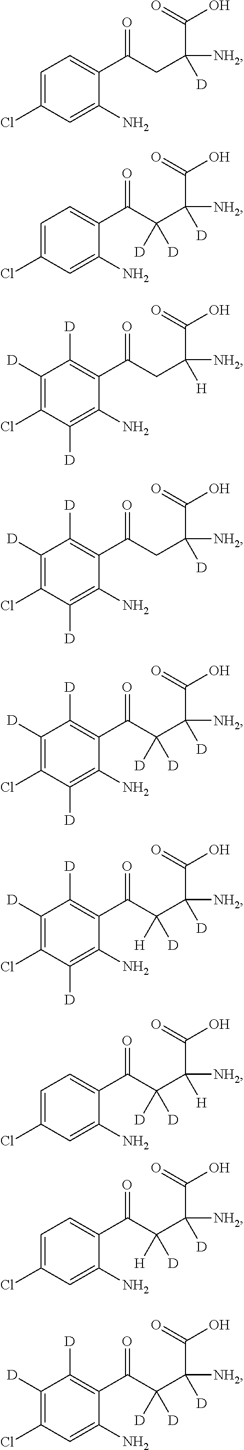

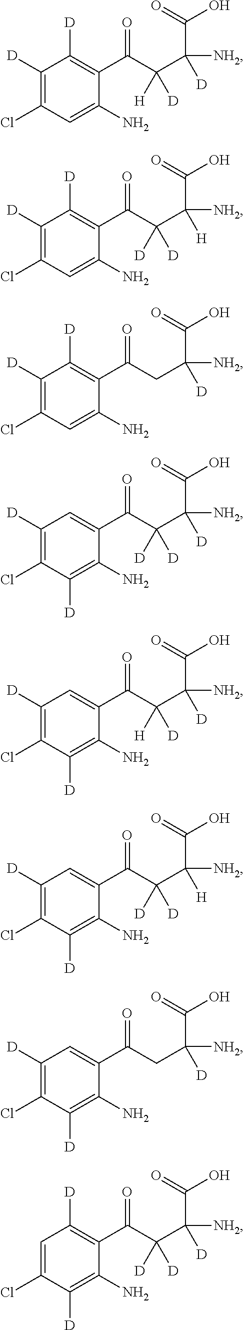

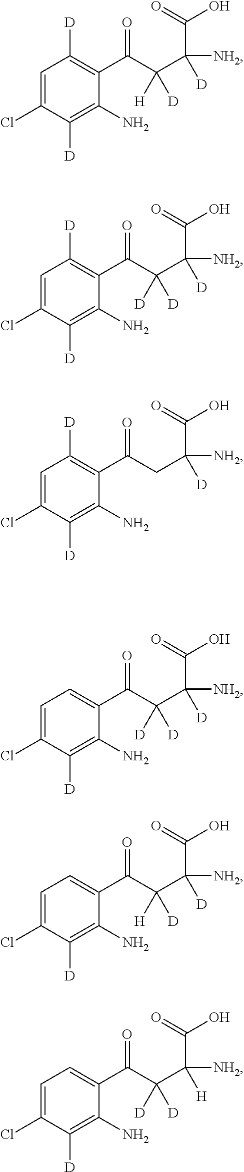 [SMILES: [2H]c1cc(Cl)c([2H])c(N)c1C(=O)C([2H])C([2H])(N)C(=O)O ; [2H]c1cc(Cl)c([2H])c(N)c1C(=O)C([2H])([2H])C([2H])(N)C(=O)O ; [2H]c1cc(Cl)c([2H])c(N)c1C(=O)CC([2H])(N)C(=O)O ; [2H]c1c(Cl)ccc(C(=O)C([2H])([2H])C([2H])(N)C(=O)O)c1N ; [2H]c1c(Cl)ccc(C(=O)C([2H])C([2H])(N)C(=O)O)c1N ; [2H]c1c(Cl)ccc(C(=O)C([2H])([2H])C(N)C(=O)O)c1N]

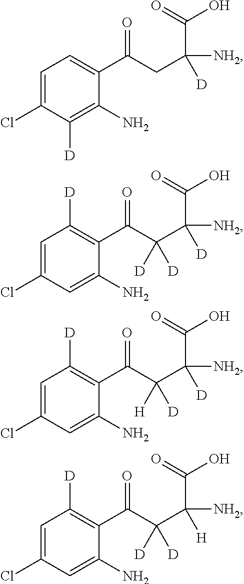

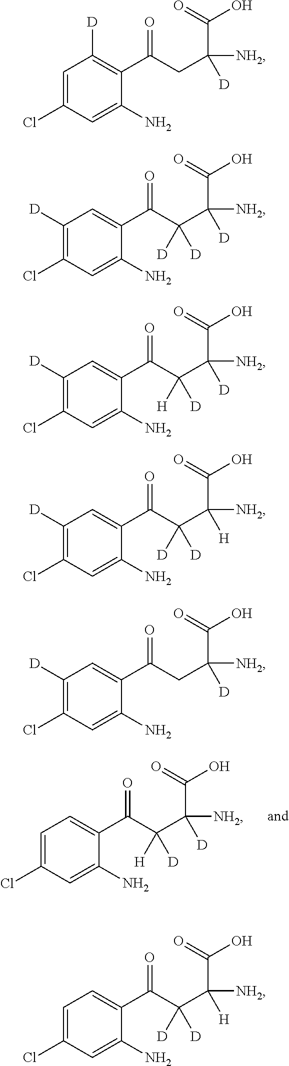

or a salt thereof.

In certain embodiments, the compound has a structural formula chosen from

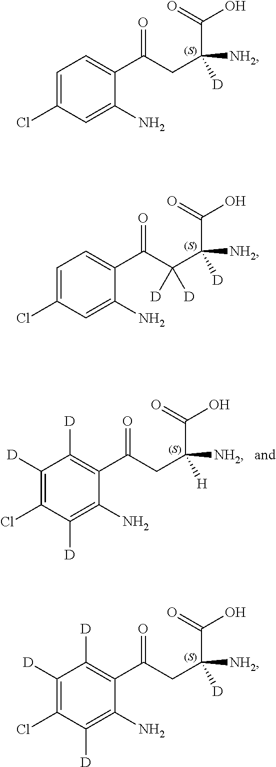

or a salt thereof.

In certain embodiments, each position represented as D has deuterium enrichment of no less than about 10%. In certain embodiments, each position represented as D has deuterium enrichment of no less than about 50%. In certain embodiments, each position represented as D has deuterium enrichment of no less than about 90%. In certain embodiments, each position represented as D has deuterium enrichment of no less than about 98%.

In certain embodiments, the compound has the structural formula:

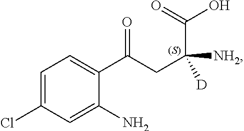

or a salt thereof.

In certain embodiments, the compound has the structural formula:

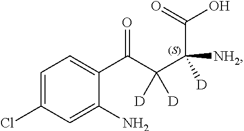

or a salt thereof.

In certain embodiments, the compound has the structural formula:

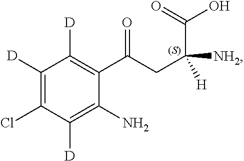

or a salt thereof.

In certain embodiments, the compound has the structural formula:

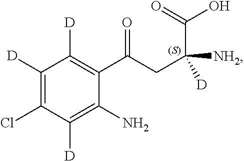

or a salt thereof.

In certain embodiments, each position represented as D has deuterium enrichment of no less than about 10%. In certain embodiments, each position represented as D has deuterium enrichment of no less than about 50%. In certain embodiments, each position represented as D has deuterium enrichment of no less than about 90%. In certain embodiments, each position represented as D has deuterium enrichment of no less than about 98%.

Also provided is a pharmaceutical composition comprising a compound as disclosed herein, together with a pharmaceutically acceptable carrier.

Also provided is a method of treatment of a NMDA receptor-mediated disorder comprising the administration of a therapeutically effective amount of a compound as disclosed herein to a patient in need thereof.

Also provided is a method of treatment of a neuropsychiatric disorder, a neurodegenerative disorder, a seizure disorder, an age-related cognitive disorder, a perinatal brain disorder, or a disorder of movement involving chorea, dyskinesia, or one or more tics, comprising the administration of a therapeutically effective amount of a compound as disclosed herein to a patient in need thereof.

Also provided is a method of treatment of a disorder chosen from Alzheimer's disease, vascular dementia, Parkinson's disease, Huntington's disease, amyotriphic lateral sclerosis, multiple sclerosis, traumatic brain injury, major depressive disorder, biopolar disorder, schizophrenia, epilepsy, hyperalgesia, neuropathic pain, migraine, Huntington's disease, tardive dyskinesia, Tourette's Syndrome, and L-DOPA associated dyskinesia, comprising the administration of a therapeutically effective amount of a compound as disclosed herein to a patient in need thereof.

In certain embodiments, the disorder is major depressive disorder.

Also provided is a method of enhancing learning, memory, or cognition in a patient, comprising the administration of a therapeutically effective amount of a compound as disclosed herein to a patient in need thereof.

In certain embodiments, the method further comprises the administration of an additional therapeutic agent.

In certain embodiments, the method results in at least one effect selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

In certain embodiments, the method results in at least two effects selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

In certain embodiments, the method effects a decreased metabolism of the compound per dosage unit thereof by at least one polymorphically-expressed metabolizing enzyme isoform in the subject, as compared to the corresponding non-isotopically enriched compound.

In certain embodiments, said compound is characterized by decreased inhibition of at least one metabolizing enzyme isoform in said subject per dosage unit thereof as compared to the non-isotopically enriched compound.

Also provided is a compound as disclosed herein for use as a medicament.

Also provided is a compound as disclosed herein for use in the manufacture of a medicament for the prevention or treatment of a disorder ameliorated by the inhibition of a NMDA receptor.

Also provided is a compound as disclosed herein for use in the manufacture of a medicament for the prevention or treatment of a neuropsychiatric disorder, a neurodegenerative disorder, a seizure disorder, an age-related cognitive disorder, a perinatal brain disorder, or a disorder of movement involving chorea, dyskinesia, or one or more tics.

Also provided is a compound as recited in any one of Claims 1-20 for use in the manufacture of a medicament for the prevention or treatment of a disorder chosen from Alzheimer's disease, vascular dementia, Parkinson's disease, Huntington's disease, amyotriphic lateral sclerosis, multiple sclerosis, traumatic brain injury, major depressive disorder, biopolar disorder, schizophrenia, epilepsy, hyperalgesia, neuropathic pain, migraine, Huntington's disease, tardive dyskinesia, Tourette's Syndrome, and L-DOPA associated dyskinesia.

In certain embodiments, the disorder is major depressive disorder.

Also provided is a compound as disclosed herein for use in the manufacture of a medicament for enhancing learning, memory, or cognition.

Also provided is a method of increasing exposure of 7-chlorokynurenic acid in the brain, comprising the administration to a subject of an amount of a compound as disclosed herein effective to increase exposure of 7-chlorokynurenic acid in the brain.

Certain compounds disclosed herein may possess useful NMDA receptor modulating activity, and may be used in the treatment or prophylaxis of a disorder in which NMDA receptors play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating NMDA receptor. Other embodiments provide methods for treating a NMDA receptor-mediated disorder in a patient in need of such treatment, comprising administering to said patient a therapeutically effective amount of a compound or composition according to the present invention. Also provided is the use of certain compounds disclosed herein for use in the manufacture of a medicament for the prevention or treatment of a disorder ameliorated by the modulation of NMDA receptors.

The compounds as disclosed herein may also contain less prevalent isotopes for other elements, including, but not limited to, ¹³C or ¹⁴C for carbon, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₃₅ or the symbol “D”, when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “NMDA receptor-mediated disorder,” refers to a disorder that is characterized by abnormal NMDA receptor activity. A NMDA receptor-mediated disorder may be completely or partially mediated by modulating NMDA receptors. In particular, a NMDA receptor-mediated disorder is one in which modulation of NMDA receptors results in some effect on the underlying disorder e.g., administration of a NMDA receptor modulator results in some improvement in at least some of the patients being treated.

The term “NMDA receptor modulator,” refers to the ability of a compound disclosed herein to alter the function of NMDA receptors. A modulator may activate the activity of a NMDA receptor, may activate or inhibit the activity of a NMDA receptor depending on the concentration of the compound exposed to the NMDA receptor, or may inhibit the activity of a NMDA receptor. Such activation or inhibition may be contingent on the occurrence of a specific event, such as activation of a signal transduction pathway, and/or may be manifest only in particular cell types. The term “modulate” or “modulation” also refers to altering the function of a NMDA receptor by increasing or decreasing the probability that a complex forms between a NMDA receptor and a natural binding partner. A modulator may increase the probability that such a complex forms between the NMDA receptor and the natural binding partner, may increase or decrease the probability that a complex forms between the NMDA receptor and the natural binding partner depending on the concentration of the compound exposed to the NMDA receptor, and or may decrease the probability that a complex forms between the NMDA receptor and the natural binding partner.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio.

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

Salts may also be prepared from suitable metal counterions such as calcium (e.g. CaOH), magnesium (e.g. Mg(OH)₂ or Mg acetate), potassium (e.g., KOH), sodium (e.g. NaOH, and zinc (e.g. Zn(OH)₂ or Zn acetate).

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art.

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Compounds discussed herein may be used in the treatment of a variety of conditions, including those modulated by the NMDA receptor. In some embodiments, the compounds discussed herein are useful in methods for treating a neuropsychiatric disorder.

In some embodiments, the compounds discussed herein are useful in methods for treating a neurodegenerative disorder. In certain embodiments, the neurodegenerative disorder is an age-related cognitive disorder or a perinatal brain disorder. In certain embodiments, the neurodegenerative disorder is Alzheimer's disease, vascular dementia, Parkinson's disease, Huntington's disease, amyotriphic lateral sclerosis, multiple sclerosis, or traumatic brain injury.

In certain embodiments, the compounds discussed herein are useful in methods for enhancing learning, memory, or cognition in a patient.

In certain embodiments, the compounds discussed herein are useful in methods of treating neurpsychiatric conditions, including those caused by neurological dysfunction. In certain embodiments, the compounds discussed herein are useful in methods of treating depression. In still other embodiments, the compounds discussed herein are useful in methods of treating major depressive disorder. In one embodiment, the major depressive disorder is biopolar disorder. In certain embodiments, the compounds discussed herein are useful in methods of treating schizophrenia. In certain embodiments, the compounds discussed herein are useful in methods of treating seizure disorders, such as epilepsy.

In yet further embodiments, the compounds discussed herein are useful in methods of treating hyperalgesia and/or neuropathic pain. In yet further embodiments, the compounds discussed herein are useful in methods of treating migraine.

In certain embodiments, the compounds discussed herein may be used in methods of treating disorders of movement, such as those involving choreas, dyskinesias, and/or tics. In certain embodiments, the movement disorder is chosen from Huntington's disease, tardive dyskinesia, and Tourette's Syndrome. In some embodiments, the compounds discussed herein may be used in methods for reducing L-DOPA associated dyskinesias.

In certain embodiments, a method of treating a NMDA receptor-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) at least one statistically-significantly improved disorder-control and/or disorder-eradication endpoint; and (4) an improved clinical effect during the treatment of the disorder, as compared to the corresponding non-isotopically enriched compound.

Examples of improved disorder-control and/or disorder-eradication endpoints, or improved clinical effects include, but are not limited to, any one or more of:
1. improvement on Hamilton Rating Scale for Depression;
2. improvement on Zung Self-Rated Depression Scale;
3. improvement on Hospital Anxiety and Depression Scale (HADS);
4. improvement on Columbia Suicide Severity Rating Scale (C-SSRS);
5. improvement on Brief Psychiatric Rating Scale;
6. improvement on Unified Huntington's Disease Rating Scale (UHDRS);
7. improvement on Patient Global Impression of Change (PGIC);
8. improvement on Clinical Global Impression of Change (CGIC);
9. improvement on Abnormal Involuntary Movement Scale (AIMS); and
10. reduced frequency, duration, or severity of seizures.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of the disorders described herein. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepam; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopeptidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, tricrynafen, chlorthalidone, furosemide, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone antagonists, and octreotide acetate; microtubule-disruptor agents, such as ecteinascidins; microtubule-stabilizing agents, such as paclitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunomide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating NMDA receptor-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of NMDA receptor-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof. Compounds as disclosed herein can also be prepared as shown in any of the following schemes and routine modifications thereof.

The following schemes can be used to practice the present invention. Any position shown as hydrogen may optionally be replaced with deuterium.

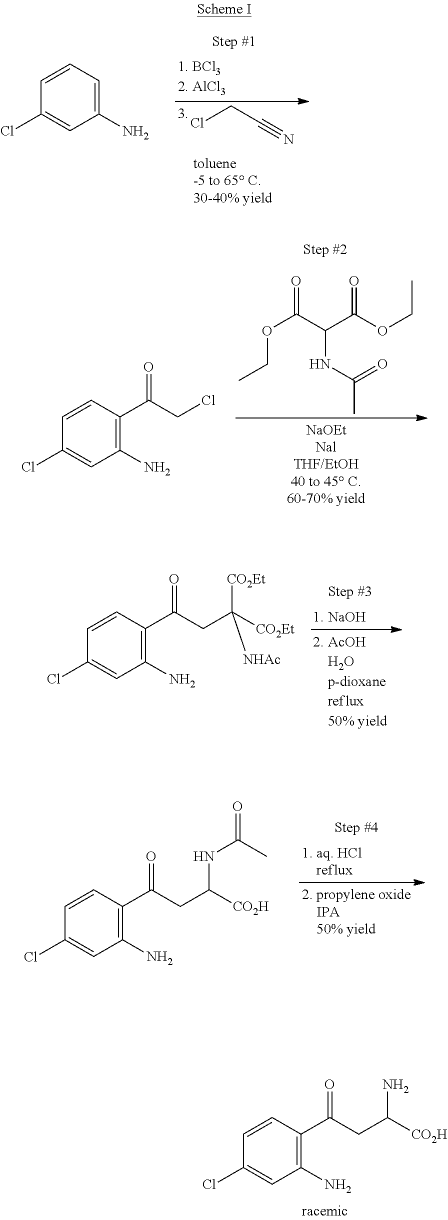

Scheme I may be used to synthesize racemic 4-chlorokynurenine.

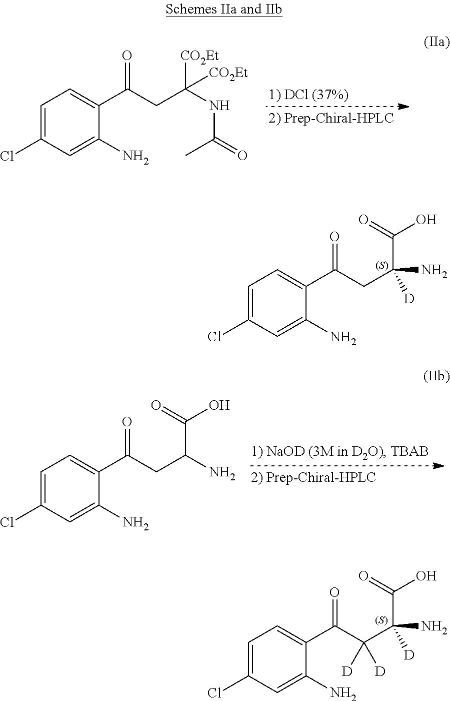

Scheme IIa and IIb may be used to synthesize deuterated derivatives of 4-chlorokynurenine from racemic 4-chlorokynurenine. Either a deuterated acid such as deuterium chloride or a deuterated base such as sodium deuteroxide may be used. Racemates may then be isolated by preparative chiral HPLC.

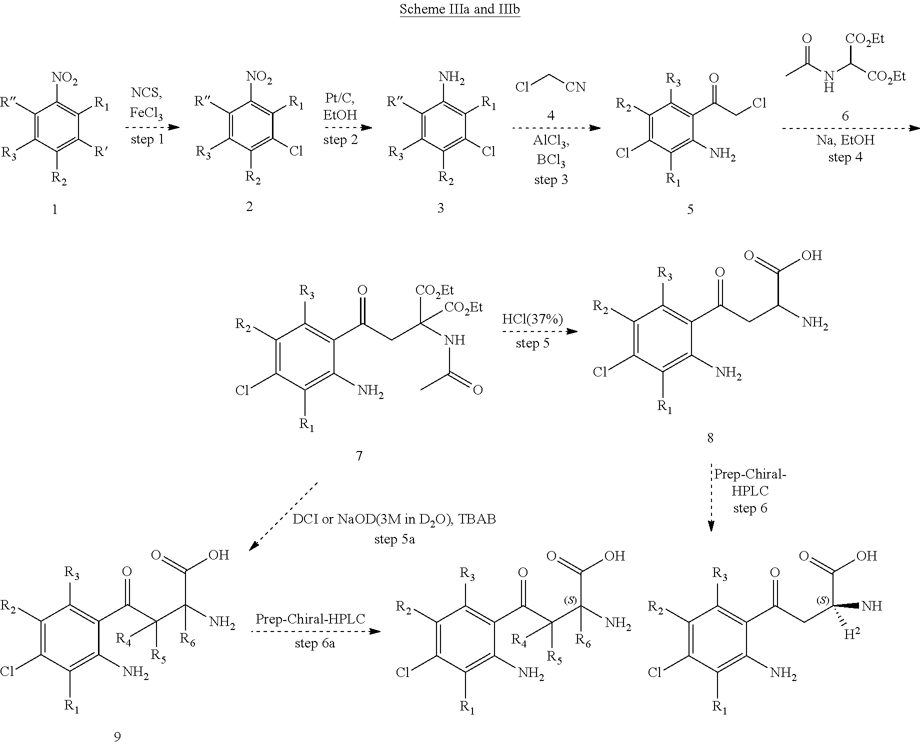

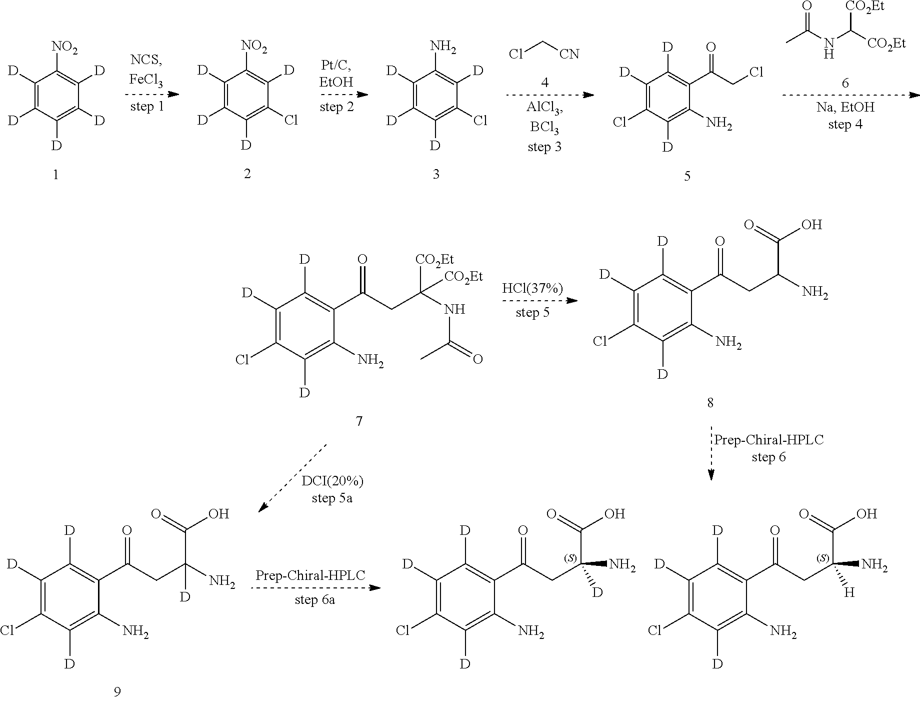

Schemes IIIa and IIIb may be used to synthesize deuterated derivatives of 4-chlorokynurenine from deuterated nitrobenzenes (in which R₁, R₂, R₃, R′, and R″ are either hydrogen or deuterium) such as d₅-nitrobenzene (IIIb). Racemates may then be isolated by preparative chiral HPLC.

For example, compound 1 is reacted with a chlorinating agent such as FeCl₃ and N-chlorosuccinamide to form a compound 2. Compound 2 is treated with an appropriate reducing agent, such as platinum on carbon and an alcohol such as ethanol, to give compound 3. Compound 3 is treated with one or more appropriate Lewis acids, such as a combination of AlCl₃ and BCl₃, in an appropriate solvent, such as 2-chloroacetonitrile, to give compound 5. Compound 5 is treated with diethyl 2-acetamidomalonate, in the presence of an sodium and an alcohol such as ethanol, to give compound 7. Compound 7 can then be used to prepare additional deuterated derivatives.

Deuterium can be incorporated to different positions synthetically, according to the synthetic procedures as shown in schemes above, by using appropriate deuterated intermediates. Deuterium can be incorporated to various positions having an exchangeable proton, such as the carboxyl O—H, via proton-deuterium equilibrium exchange. For example, to introduce deuterium at R₁ this proton may be replaced with deuterium selectively or non-selectively through a proton-deuterium exchange method known in the art.

The invention is further illustrated by the following examples. The following compounds can generally be made using the methods described above. It is expected that these compounds when made will have activity similar to those described in the examples above.

The following abbreviations may be employed in the Examples and elsewhere herein:
DMA=dimethylacetamide
DMF=dimethylformamide
DMSO=dimethyl sulfoxide
DCM=dichloromethane
EA=ethyl acetate
L=liter
mL=milliliter
μL-microliter
g=gram(s)
mg=milligram(s)
mol=moles
mmol=millimole(s)
h or hr=hour(s)
min=minute(s)
Equiv=equivalent(s)
H₂=hydrogen
Ar=argon
N₂=nitrogen
RT or R.T.=room temperature
AT=ambient temperature
Aq.=aqueous
HPLC=high performance liquid chromatography
HPLC R,=HPLC retention time
LC/MS=high performance liquid chromatography/mass spectrometry
MS or Mass Spec=mass spectrometry
NMR=nuclear magnetic resonance
NMR spectral data: s=singlet; d=doublet; m=multiplet; br=broad; t=triplet
mp=melting point

All IUPAC names were generated using PerkinElmer®'s ChemDraw.

### EXAMPLES

### Example 1—Comparative

### (2S)-2-amino-4-(2-amino-4-chlorophenyl)-4-oxobutanoic acid

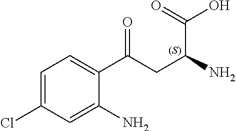

### Step 1: 1-(2-amino-4-chlorophenyl)-2-chloroethan-1-one

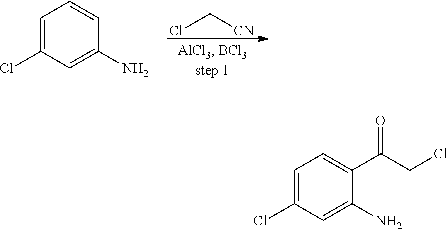

To a solution of BCl₃ (317 mL, 1.10 equiv) in toluene (500 mL) was added 3-chloroaniline (36.6 g, 286.90 mmol, 1.00 equiv) at −5 to 3° C. Then AlCl₃ (38 g, 1.00 equiv) was added. After 3 min, 2-chloroacetonitrile (28.1 g, 372.20 mmol, 1.30 equiv) was added. The resulting solution was stirred at 70° C. overnight. Then the mixture was added to 1N HCl (500 mL) over 30 min at 50° C. The solution was extracted with DCM (3×300 mL). The organic layers were concentrated under vacuum. Hexane (200 mL) was added to the mixture. The solids were filtered out to afford 28.6 g (49%) of 1-(2-amino-4-chlorophenyl)-2-chloroethan-1-one as a yellow solid. LC-MS: m/z=204 [M+H]⁺.

### Step 2: 1,3-diethyl 2-[2-(2-amino-4-chlorophenyl)-2-oxoethyl]-2-acetamidopropanedioate

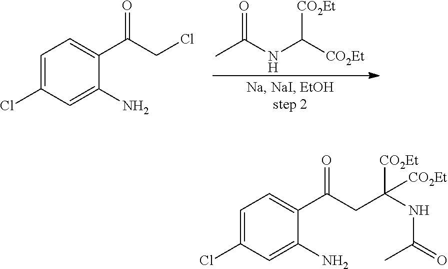

Na (3.94 g, 1.20 equiv) was added to ethanol (300 mL). The solution was stirred for 1 h at room temperature. 1,3-diethyl 2-acetamidopropanedioate (32.6 g, 150.08 mmol, 1.05 equiv) (step 1) was added. The reaction solution was stirred for 20 min at room temperature. Then 1-(2-amino-4-chlorophenyl)-2-chloroethan-1-one (29 g, 142.12 mmol, 1.00 equiv), NaI (3.21 g, 0.15 equiv), THF (150 mL) were added. The resulting solution was stirred for 3 h at 50° C. The mixture was added to ice/water (600 mL). The resulting solution was extracted with ethyl acetate (3×200 mL), and the organic layers were combined, washed with brine (2×200 mL), dried over anhydrous sodium sulfate, and concentrated under vacuum. The crude product was dissolved in ethyl acetate (30 mL). Hexane (200 mL) was added to the mixture. The solids were filtered out to afford 28 g (51%) of 1,3-diethyl 2-[2-(2-amino-4-chlorophenyl)-2-oxoethyl]-2-acetamidopropanedioate as a light brown solid. LC-MS: m/z=385 [M+H]⁺.

### Step 3: 2-amino-4-(2-amino-4-chlorophenyl)-4-oxobutanoic acid

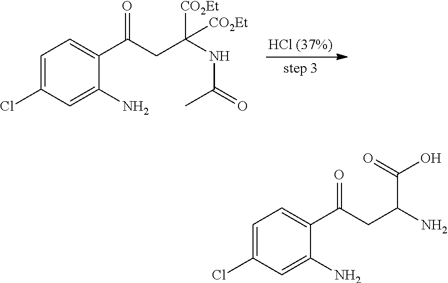 [SMILES: CCOC(=O)C(CC(=O)c1ccc(Cl)cc1N)(NC(C)=O)C(=O)OCC ; Nc1cc(Cl)ccc1C(=O)CC(N)C(=O)O]

A solution of 1,3-diethyl 2-[2-(2-amino-4-chlorophenyl)-2-oxoethyl]-2-acetamidopropanedioate (15 g, 39.06 mmol, 1.00 equiv) (step 2) in hydrogen chloride (100 mL) was stirred for 3 h at 110° C. The mixture was concentrated under vacuum. Then the solid was dissolved in H₂O (20 mL). The pH value of the aqueous solution was adjusted to 7 with NaOH (2 M). The solids were filtered out to afford 8 g (85%) of 2-amino-4-(2-amino-4-chlorophenyl)-4-oxobutanoic acid as a yellow solid. LC-MS: m/z=243 [M+H]⁺.

### Step 4: methyl (2S)-2-amino-4-(2-amino-4-chlorophenyl)-4-oxobutanoate

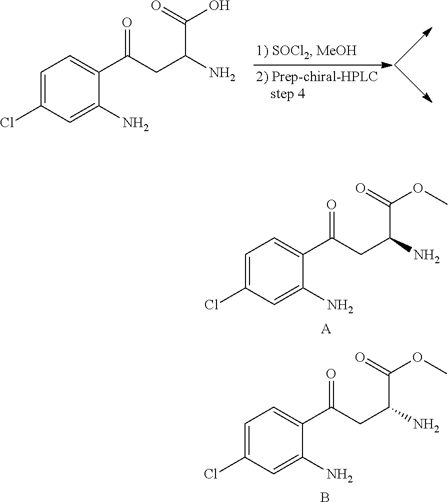

To a solution of thionyl chloride (12 mL, 5.00 equiv) in MeOH (100 mL) was added 2-amino-4-(2-amino-4-chlorophenyl)-4-oxobutanoic acid (8 g, 33.06 mmol, 1.00 equiv). The resulting solution was stirred for 2 h at 70° C. Then the resulting solution was concentrated under vacuum to remove MeOH. The solution of NaHCO₃ was added to the residue. The resulting solution was extracted with EA (3×50 mL). The organic layers were dried over anhydrous sodium sulfate and concentrated under vacuum to afford 8 g (95%) of racemic mixture as a yellow solid. The racemic mixture was purified by Prep-SFC with the following conditions (Prep SFC80-2): Column, CHIRALPAK IC, 2*25 cm, 5 um; mobile phase, CO₂ (60%), IPA (0.1% 2mMNH₃MeOH) (40%); Detector, UV 220 nm. This resulted in (A): 3.5 g (44%) of methyl (2S)-2-amino-4-(2-amino-4-chlorophenyl)-4-oxobutanoate as a light yellow solid and (B): 3.5 g (44%) methyl (2R)-2-amino-4-(2-amino-4-chlorophenyl)-4-oxobutanoate as a light yellow solid. LC-MS: m/z=257 [M+H]⁺.

### Step 5: (2S)-2-amino-4-(2-amino-4-chlorophenyl)-4-oxobutanoic acid

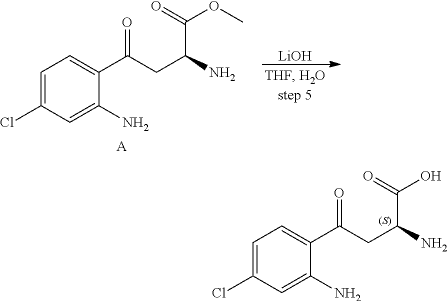

To a solution of methyl (2S)-2-amino-4-(2-amino-4-chlorophenyl)-4-oxobutanoate (3.5 g, 13.64 mmol, 1.00 equiv) (step 4, compound (A)) in THF (30 mL) and H₂O (10 mL) was added LiOH (980 mg, 40.92 mmol, 3.00 equiv). The resulting solution was stirred for 3 h at room temperature. The reaction progress was monitored by LCMS. The resulting solution was concentrated under vacuum to remove THF. Then the pH value of the solution was adjusted to 4 with HCl (3 M), and the solids were filtered out. The filtrate was lyophilized. The crude product was purified by Flash-Prep-HPLC with the following conditions (IntelFlash-1): Column, C18 silica gel; mobile phase, CH₃CN/H₂O=1:9 increasing to CH₃CN/H₂O=2:3 within 20 min; Detector, UV 220 nm. This resulted in 2.10 g (63%) of (2S)-2-amino-4-(2-amino-4-chlorophenyl)-4-oxobutanoic acid as a white solid. ¹H NMR (400 MHz, D₂O) δ: 7.83-7.80 (m, 1H), 7.19-7.15 (m, 2H), 4.38-4.35 (m, 1H), 3.77-3.55 (m, 2H). LC-MS: m/z=243 [M+H]⁺.

### Example 2—Comparative

### (2R)-2-amino-4-(2-amino-4-chlorophenyl)-4-oxobutanoic acid

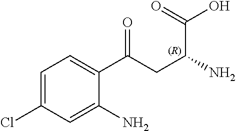

Step 1: (2R)-2-amino-4-(2-amino-4-chlorophenyl)-4-oxobutanoic acid

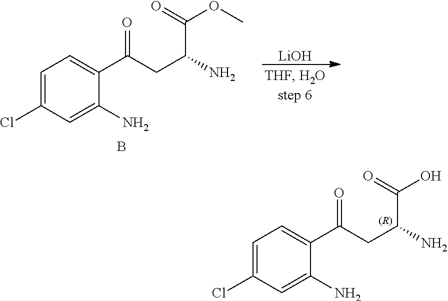

To a solution of methyl (2R)-2-amino-4-(2-amino-4-chlorophenyl)-4-oxobutanoate (3.5 g, 13.64 mmol, 1.00 equiv) (Example 1, step 4, compound (B)) in THF (30 mL) and H₂O (10 mL) was added LiOH (980 mg, 40.92 mmol, 3.00 equiv). The resulting solution was stirred for 3 h at room temperature. The reaction progress was monitored by LCMS. The resulting solution was concentrated under vacuum to remove THF. Then the pH value of the solution was adjusted to 4 with HCl (3 M), and the solids were filtered out. The filtrate was lyophilized. The crude product was purified by Flash-Prep-HPLC with the following conditions (IntelFlash-1): Column, C18 silica gel; mobile phase, CH₃CN/H₂O=1:9 increasing to CH₃CN/H₂O=2:3 within 20 min; Detector, UV 220 nm. This resulted in 2.10 g (63%) of (2R)-2-amino-4-(2-amino-4-chlorophenyl)-4-oxobutanoic acid as a white solid. ¹H NMR (400 MHz, D₂O) δ: 7.68-7.66 (m, 1H), 6.93-6.88 (m, 2H), 4.73-4.35 (m, 1H), 3.74-3.51 (m, 2H). LC-MS: m/z=243 [M+H]⁺.

### Example 3

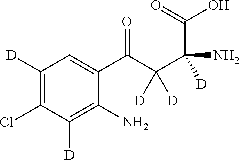 [SMILES: [2H]c1cc(C(=O)C([2H])([2H])[C@]([2H])(N)C(=O)O)c(N)c([2H])c1Cl]

### Step 1: 2-amino-4-[2-amino-4-chloro(3,5-²H₂)phenyl]-4-oxo(²H₃)butanoic acid

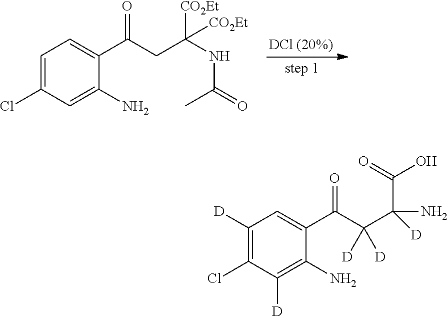 [SMILES: CCOC(=O)C(CC(=O)c1ccc(Cl)cc1N)(NC(C)=O)C(=O)OCC ; [2H]c1cc(C(=O)C([2H])([2H])C([2H])(N)C(=O)O)c(N)c([2H])c1Cl]

A solution of 1,3-diethyl 2-[2-(2-amino-4-chlorophenyl)-2-oxoethyl]-2-acetamidopropanedioate (28 g, 72.76 mmol, 1.00 equiv) (Example 1, Step 2) in DCl (20% in D₂O) (70 mL) was stirred for 12 h at 110° C. The mixture was concentrated under vacuum. Then the residue was dissolved in DC1. The final reaction mixture was irradiated with microwave radiation for 2 h at 160° C. The crude product was purified by Flash-Prep-HPLC with the following conditions (IntelFlash-1): Column, C18 silica gel; mobile phase, CH₃CN/H₂O=1:9 increasing to CH₃CN/H₂O=2:3 within 20 min; Detector, UV 220 nm. This resulted in 6.54 g (36%) of 2-amino-4-[2-amino-4-chloro(3,5-²H₂)phenyl]-4-oxo(²H₃)butanoic acid as a yellow solid. LC-MS: m/z=248 [M+H]⁺.

### Step 2: (²H₃)methyl(2S)-2-amino-4-[2-amino-4-chloro(3,5-²H₂)phenyl]-4-oxo(²H₃)butanoate

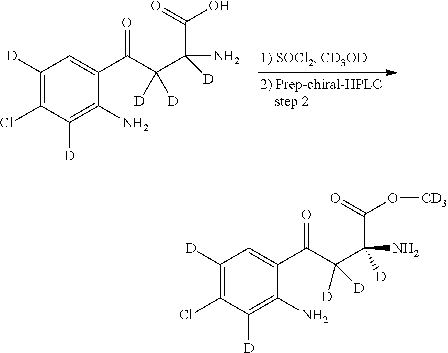 [SMILES: [2H]c1cc(C(=O)C([2H])([2H])C([2H])(N)C(=O)O)c(N)c([2H])c1Cl ; [2H]c1cc(C(=O)C([2H])([2H])[C@]([2H])(N)C(=O)OC([2H])([2H])[2H])c(N)c([2H])c1Cl]

To a solution of thionyl chloride (15.6 g, 5.00 equiv) in CD₃OD (70 mL) was added 3-amino-1-[2-amino-4-chloro(3,5-²H₂)cyclohexyl](2,2,3-²H₃)butane-1,4,4-triol (6.54 g, 26.40 mmol, 1.00 equiv). The resulting solution was stirred for 2 h at 70° C. Then the resulting solution was concentrated under vacuum to remove CD₃OD. The solution of NaHCO₃ was added to the residue. The resulting solution was extracted with ethyl acetate (3×50 mL). The organic layers were dried over anhydrous sodium sulfate and concentrated under vacuum to afford 6.5 g (93%) of racemic mixture as a yellow solid. The racemic mixture was purified by Prep-SFC with the following conditions (Prep SFC80-2): Column, CHIRALPAK IC, 2*25 cm, 5 um; mobile phase, CO₂ (60%), IPA (0.1% 2mMNH₃ MeOH) (40%); Detector, UV 220 nm. This resulted in 2.3 g of (²H₃)methyl (2S)-2-amino-4-[2-amino-4-chloro(3,5-²H₂)phenyl]-4-oxo(²H₃)butanoate as a yellow solid. LC-MS: m/z=265 [M+H]⁺

### Step 3: (2S)-2-amino-4-[2-amino-4-chloro(3,5-²H₂)phenyl]-4-oxo(²H₃)butanoic acid

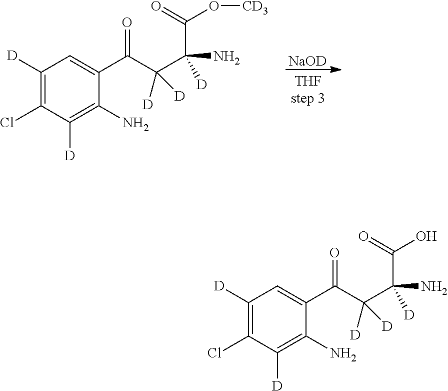

To a solution of (²H₃)methyl (2S)-2-amino-4-[2-amino-4-chloro(3,5-²H₂)phenyl]-4-oxo(²H₃)butanoate (400 mg, 1.51 mmol, 1.00 equiv) (step 2) in THF (10 mL) was added NaOD (in D₂O, 3M) (1.54 mL, 3.00 equiv). The resulting solution was stirred for 3 h at room temperature. The reaction progress was monitored by LCMS. The resulting solution was concentrated under vacuum to remove THF. Then the pH value of the solution was adjusted to 4 with DC1 (20% in D₂O). The mixture was lyophilized. The crude product was purified by Flash-Prep-HPLC with the following conditions (IntelFlash-1): Column, C18 silica gel; mobile phase, CH₃CN/H₂O=1:9 increasing to CH₃CN/H₂O=2:3 within 20 min; Detector, UV 220 nm. This resulted in 170 mg (45%) of (2S)-2-amino-4-[2-amino-4-chloro(3,5-²H₂)phenyl]-4-oxo(²H₃)butanoic acid as a white solid. ¹H NMR (400 MHz, D₂O) δ: 7.83-7.81 (m, 1H). LC-MS: m/z=248 [M+H]⁺.

### Example 4

### (2S)-2-amino-4-[2-amino-4-chloro(3,5-²H₂)phenyl]-4-oxo(2-2H)butanoic acid

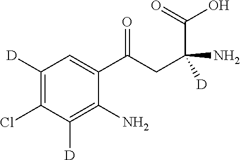 [SMILES: [2H]c1cc(C(=O)C[C@]([2H])(N)C(=O)O)c(N)c([2H])c1Cl]

### Step 1: (2S)-2-amino-4-[2-amino-4-chloro(3,5-²H₂)phenyl]-4-oxo(2-²H)butanoic acid

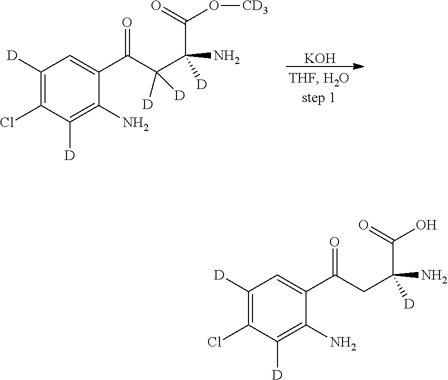 [SMILES: [2H]c1cc(C(=O)C([2H])([2H])[C@]([2H])(N)C(=O)OC([2H])([2H])[2H])c(N)c([2H])c1Cl ; "KOH" ; [2H]c1cc(C(=O)C[C@]([2H])(N)C(=O)O)c(N)c([2H])c1Cl]

To a solution of (²H₃)methyl (2S)-2-amino-4-[2-amino-4-chloro(3,5-²H₂)phenyl]-4-oxo(²H₃)butanoate (1 g, 3.78 mmol, 1.00 equiv) (Example 3) in THF (30 mL) and H₂O (10 mL) was added KOH (636 mg, 11.33 mmol, 3.00 equiv). The resulting solution was stirred for 3 h at room temperature. The reaction progress was monitored by LCMS. The resulting solution was concentrated under vacuum to remove THF. Then the pH value of the solution was adjusted to 4 with HCl (2 M). The mixture was lyophilized. The crude product was purified by Flash-Prep-HPLC with the following conditions (IntelFlash-1): Column, C18 silica gel; mobile phase, CH₃CN/H₂O=1:9 increasing to CH₃CN/H₂O=2:3 within 20 min; Detector, UV 220 nm. This resulted in 560 mg (60%) of (2S)-2-amino-4-[2-amino-4-chloro(3,5-²H₂)phenyl]-4-oxo(2-2H)butanoic acid as a white solid. ¹H NMR (400 MHz, D₂O) δ: 7.85 (s, 1H), 3.82-3.72 (m, 2H). LC-MS: m/z=246 [M+H]⁺.

### Example 5

### (2S)-2-amino-4-[2-amino-4-chloro(²H₃)phenyl]-4-oxo(2-2H)butanoic acid

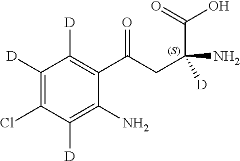

### Step 1: 1-[2-amino-4-chloro(²H₃)phenyl]-2-chloroethan-1-one

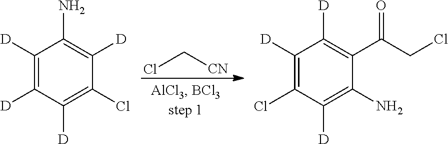

To a solution of BCl₃ (218 mL, 1.10 equiv) in toluene (300 mL) was added 3-chloro(²H₄)aniline (26 g, 197.57 mmol, 1.00 equiv) at −5 to 3° C. Then AlCl₃ (26.2 g, 1.00 equiv) was added. After 3 min, 2-chloroacetonitrile (19.4 g, 256.96 mmol, 1.30 equiv) was added. The resulting solution was stirred at 70° C. overnight. Then the mixture was added to 1N HCl (500 mL) over 30 min at 50° C. The solution was extracted with DCM (3×300 mL). The organic layers were concentrated under vacuum. Hexane (200 mL) was added to the mixture. The solids were filtered out to afford 10 g (24%) of 1-[2-amino-4-chloro(²H₃)phenyl]-2-chloroethan-1-one as a brown solid.

### Step 2: 1,3-diethyl2-[2-[2-amino-4-chloro(²H₃)phenyl]-2-oxoethyl]-2-acetamidopropanedioate

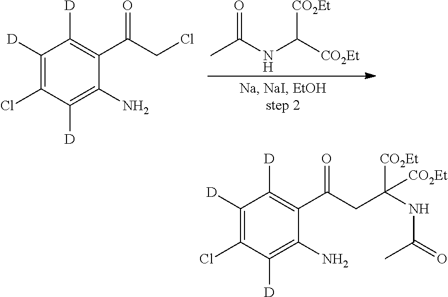

Na (938 mg 1.20 equiv) was added ethanol (100 mL). The solution was stirred for 1 h at room temperature. 1,3-diethyl 2-acetamidopropanedioate (7.7 g, 35.45 mmol, 1.05 equiv) was added. The reaction solution was stirred for 20 min at room temperature. Then 1-[2-amino-4-chloro(²H₃)phenyl]-2-chloroethan-1-one (7 g, 33.80 mmol, 1.00 equiv) (step 1), NaI (765 mg, 0.15 equiv), THF (50 mL) were added. The resulting solution was stirred for 3 h at 50° C. The mixture was added to ice/water (200 mL). The resulting solution was extracted with ethyl acetate (3×100 MI), and the organic layers were combined, washed with brine (2×100 mL), dried over anhydrous sodium sulfate, and concentrated under vacuum to afford 210 g (76%) of 1,3-diethyl 2-[2-[2-amino-4-chloro(²H₃)phenyl]-2-oxoethyl]-2-acetamidopropanedioate as brown oil.

### Step 3: 2-amino-4-[2-amino-4-chloro(²H₃)phenyl]-4-oxo(²H₃)butanoic acid

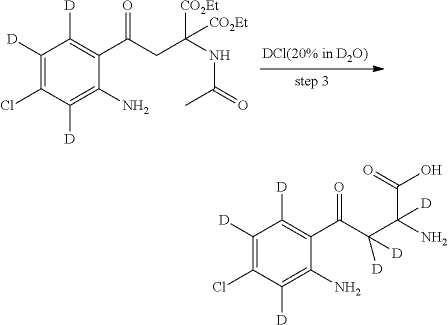 [SMILES: [2H]c1c([2H])c(C(=O)CC(NC(C)=O)(C(=O)OCC)C(=O)OCC)c(N)c([2H])c1Cl ; [2H]c1c([2H])c(C(=O)C([2H])([2H])C([2H])(N)C(=O)O)c(N)c([2H])c1Cl]

A solution of 1,3-diethyl 2-[2-[2-amino-4-chloro(²H₃)phenyl]-2-oxoethyl]-2-acetamidopropanedioate (10 g, 25.78 mmol, 1.00 equiv) in DCl (50 mL) was stirred for 12 h at 110° C. The mixture was concentrated under vacuum. The crude product was purified by Flash-Prep-HPLC with the following conditions (IntelFlash-1): Column, C18 silica gel; mobile phase, CH₃CN/H₂O=1:9 increasing to CH₃CN/H₂O=2:3 within 20 min; Detector, UV 220 nm. This resulted in 3.1 g (48%) of 2-amino-4-[2-amino-4-chloro(²H₃)phenyl]-4-oxo(²H₃)butanoic acid as a light yellow solid.

### Step 4: (²H₃)methyl (2S)-2-amino-4-[2-amino-4-chloro(²H₃)phenyl]-4-oxo(²H₃)butanoate

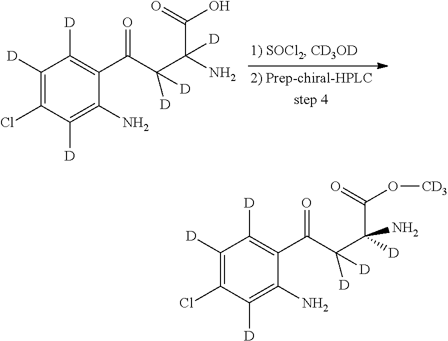 [SMILES: [2H]c1c([2H])c(C(=O)C([2H])([2H])C([2H])(N)C(=O)O)c(N)c([2H])c1Cl ; [2H]c1c([2H])c(C(=O)C([2H])([2H])[C@]([2H])(N)C(=O)OC([2H])([2H])[2H])c(N)c([2H])c1Cl]

To a solution of thionyl chloride (4.5 mL, 5.00 equiv) in CD₃OD (35 mL) was added 2-amino-4-[2-amino-4-chloro(²H₃)phenyl]-4-oxo(²H₃)butanoic acid (3.1 g, 12.47 mmol, 1.00 equiv) (step 3). The resulting solution was stirred for 2 h at 70° C. Then the resulting solution was concentrated under vacuum to remove CD₃OD. The solution of NaHCO₃ was added to the residue. The resulting solution was extracted with ethyl acetate (3×20 mL). The organic layers were dried over anhydrous sodium sulfate and concentrated under vacuum to afford 3.0 g (91%) of racemic mixture as a yellow solid. The racemic mixture was purified by Prep-SFC with the following conditions (Prep SFC80-2): Column, CHIRALPAK IC, 2*25 cm, 5 um; mobile phase, CO₂ (60%), IPA (0.1%2mMNH₃-MeOH) (40%); Detector, UV 220 nm. This resulted in 1.3 g (43%) of (²H₃)methyl (2S)-2-amino-4-[2-amino-4-chloro(²H₃)phenyl]-4-oxo(²H₃)butanoate as a yellow solid.

### Step 5: (2S)-2-amino-4-[2-amino-4-chloro(²H₃)phenyl]-4-oxo(2-2H)butanoic acid

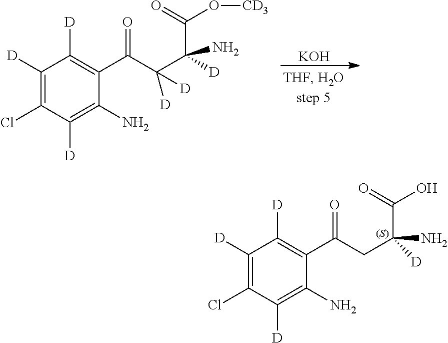

To a solution of (²H₃)methyl (2S)-2-amino-4-[2-amino-4-chloro(²H₃)phenyl]-4-oxo (²H₃)butanoate (1.04 g, 3.91 mmol, 1.00 equiv) (step 4) in THF (6 mL) and water (2 mL) was added KOH (660 mg, 11.76 mmol, 3.00 equiv). The resulting solution was stirred for 3 h at room temperature. The reaction progress was monitored by LCMS. The resulting solution was concentrated under vacuum to remove THF. Then the pH value of the solution was adjusted to 4 with HCl (3 M), and the solids were filtered out. The filtrate was lyophilized. The crude product was purified by Flash-Prep-HPLC with the following conditions (IntelFlash-1): Column, C18 silica gel; mobile phase, CH₃CN/H₂O=1:9 increasing to CH₃CN/H₂O=2:3 within 20 min; Detector, UV 220 nm. This resulted in 600 mg (62%) of (2S)-2-amino-4-[2-amino-4-chloro(²H₃)phenyl]-4-oxo(2-2H)butanoic acid as a white solid. ¹H NMR (400 MHz, D₂O) δ: 3.73-3.73 (m, 2H). LC-MS: m/z=247 [M+H]⁺.

### Example 6

### 7-chloro-4-oxo-1,4-dihydro(6,8-²H₂)quinoline-2-carboxylic acid

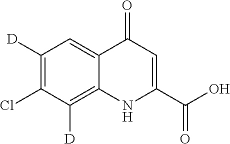 [SMILES: [2H]c1cc2c(=O)cc(C(=O)O)[nH]c2c([2H])c1Cl]

### Step 1: 3-chloro(2,4,6-²H₃)aniline

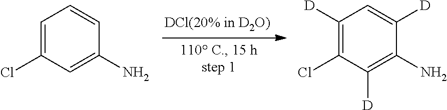 [SMILES: Nc1cccc(Cl)c1 ; [2H]c1cc([2H])c(Cl)c([2H])c1N]

A solution of 3-chloroaniline (3.0 g, 23.52 mmol, 1.00 equiv) in DCl (20% in D₂O, 10.0 mL) was placed into a sealed tube and stirred at 110° C. overnight. The reaction progress was monitored by LCMS and the result showed that the Deuterium Content (%) of the product was less than 95%. The residual DCl of the reaction solution was evaporated and the fresh DCl (10.0 mL) was introduced to the reaction system again. The resulting mixture was stirred for another 5 hrs at 110° C. Since the Deuterium Content (%) of the product was above 95%, the pH value of the reaction solution was adjusted to 9 by using 10% sodium hydroxide. Then the product was extracted with ethyl acetate (3×20.0 mL). The organic layers combined and washed with saturated brine (1×20.0 mL), dried over anhydrous sodium sulfate, and concentrated under vacuum to afford 3.0 g (98%) of 3-chloro(2,4,6-²H₃)aniline as a light yellow liquid.

### Step 2: 1,4-diethyl (2E)-2-[[3-chloro(2,4,6-²H₃) phenyl]imino]butanedioate

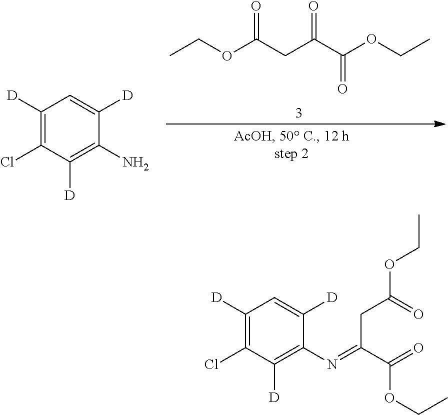 [SMILES: CCOC(=O)CC(=O)C(=O)OCC ; [2H]c1cc([2H])c(Cl)c([2H])c1N ; [2H]c1cc([2H])c(/N=C(\CC(=O)OCC)C(=O)OCC)c([2H])c1Cl]

To a solution of 3-chloro(2,4,6-²H₃) aniline (3.1 g, 23.74 mmol, 1.00 equiv) (step 1) in toluene (20.0 mL) was added 1,4-diethyl 2-oxobutanedioate (5.38 g, 28.59 mmol, 1.20 equiv). The resulting solution was stirred for 6 h at 45° C. The reaction mixture was cooled to room temperature and concentrated under vacuum to remove the toluene. The residue liquid was dissolved with methanol and applied onto a silica gel column with ethyl acetate/petroleum ether (1:10). The collected fractions were combined and concentrated under vacuum to afford 1.83 g (26%) of 1,4-diethyl (2E)-2-[[3-chloro(2,4,6-²H₃) phenyl]imino]butanedioate as light yellow oil.

### Step 3: ethyl 7-chloro-4-oxo-1,4-dihydro(6,8-²H₂)quinoline-2-carboxylate

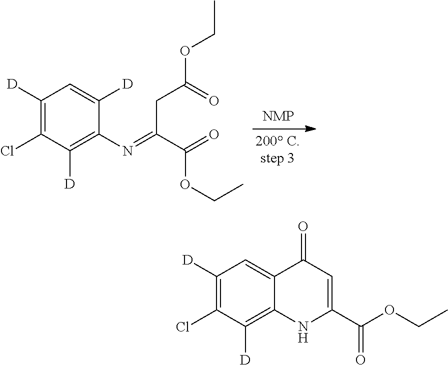 [SMILES: [2H]c1cc([2H])c(/N=C(\CC(=O)OCC)C(=O)OCC)c([2H])c1Cl ; [2H]c1cc2c(=O)cc(C(=O)OCC)[nH]c2c([2H])c1Cl]

A solution of 1,4-diethyl (2E)-2-[[3-chloro(2,4,6-²H₃)phenyl]imino]butanedioate (1.83 g, 6.08 mmol, 1.00 equiv) (step 2) in NMP (6 mL) was introduced into a 40 mL sealed tube. The reaction mixture was irradiated with microwave radiation for 25 minute at 220° C. The reaction mixture was then cooled to room temperature and quenched by water/ice. The resulted solution was extracted with ethyl acetate (3×20 mL), and the combined organic layer was washed with saturated brine (1×20 mL), dried over anhydrous sodium sulfate and concentrated under vacuum. The crude product was re-crystallized from EA: N-hexane in the ratio of 1:4. The precipitated solid were filtered off to afford 790 mg (51%) of ethyl 7-chloro-4-oxo-1,4-dihydro(6,8-²H₂)quinoline-2-carboxylate as a brown solid.

### Step 4: 7-chloro-4-oxo-1,4-dihydro(6,8-²H₂)quinoline-2-carboxylic acid

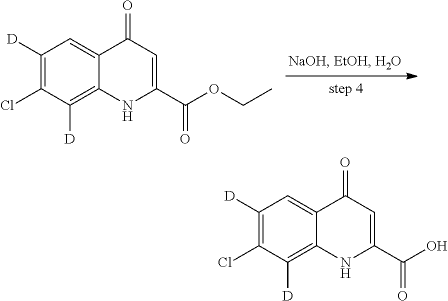 [SMILES: [2H]c1cc2c(=O)cc(C(=O)OCC)[nH]c2c([2H])c1Cl ; [2H]c1cc2c(=O)cc(C(=O)O)[nH]c2c([2H])c1Cl]

To a solution of ethyl 7-chloro-4-oxo-1,4-dihydro(6,8-²H₂)quinoline-2-carboxylate (772 mg, 3.04 mmol, 1.00 equiv) (step 3) in ethanol (20 mL) was added a solution of sodium hydroxide (365 mg, 9.12 mmol, 3.00 equiv) in water (7 mL). The resulting solution was stirred for 4 h at room temperature. The resulting mixture was concentrated under vacuum and the residue was dissolved into a small amount of water and the pH value of the solution was adjusted to 4 with hydrogen chloride (1 mol/L). The precipitated solids were collected by filtration and oven dried to afford 400 mg of product which was contained another structural isomer need to be separated. The crude product was purified by Prep-HPLC with the following conditions: Column, XBridge Prep C18 OBD Column, 19×150 mm 5 um; mobile phase, Waters(0.05% NH₃H₂O) and ACN (2.0% ACN up to 12.0% in 10 min); Detector, UV 254/220 nm. This resulted in 150 mg (22%) of 7-chloro-4-oxo-1,4-dihydro(6,8-²H₂)quinoline-2-carboxylic acid as a white solid. ¹H NMR (400 MHz, Methanol-d₄) δ: 8.22 (s, 1H), 6.95 (s, 1H).

### Example 7

### 7-chloro-4-oxo-1,4-dihydro(5,6,8-²H₃)quinoline-2-carboxylic acid

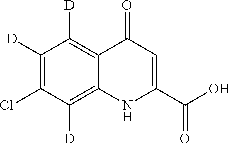 [SMILES: [2H]c1c(Cl)c([2H])c2[nH]c(C(=O)O)cc(=O)c2c1[2H]]

### Step 1: 1-chloro-3-nitro(²H₄)benzene

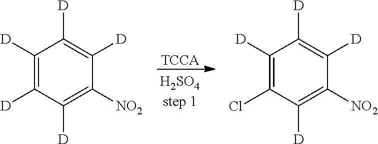

To a magnetically stirred solution of 1-nitro(²H₅)benzene (50.0 g, 390.62 mmol, 1.00 equiv) in 98% H₂SO₄ (200 mL) was added TCCA (30.68 g, 132.81 mmol, 0.34 equiv). The resulting solution was stirred for 3 h at 80° C. The reaction mixture was cooled to room temperature, and then quenched by the addition of water/ice. The resulting solution was extracted with of ethyl acetate and the organic layers combined. The resulting mixture was washed with water, saturated brine, dried over anhydrous sodium sulfate and concentrated under vacuum. The residue was applied onto a silica gel column with ethyl acetate/petroleum ether (1:50). This resulted in 60.0 g (95%) of 1-chloro-3-nitro(²H₄)benzene as light yellow oil.

### Step 2: 3-chloro(²H₄)aniline

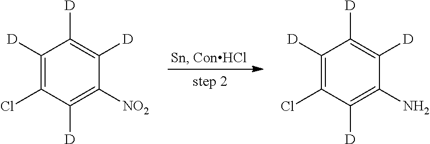 [SMILES: [2H]c1c([2H])c(Cl)c([2H])c([N+](=O)[O-])c1[2H] ; "Sn, Con·HCl" ; [2H]c1c([2H])c(N)c([2H])c(Cl)c1[2H]]

To a solution of 1-chloro-3-nitro(²H₄)benzene (54 g, 334.20 mmol, 1.00 equiv) (step 1) in Con.HCl (400 mL) was added Sn powder (118 g, 3.00 equiv) in several batches. The resulting solution was stirred for 15 h at room temperature. The pH value of the solution was adjusted to 9 with sodium hydroxide (6 mol/L). The precipitated solids were filtered out and washed with ethyl acetate. The resulting solution was extracted three times with ethyl acetate, the organic layers were combined and washed with saturated brine, dried over anhydrous sodium sulfate and concentrated under vacuum. The residue was applied onto a silica gel column with ethyl acetate/petroleum ether (1:3). This resulted in 24 g (55%) of 3-chloro(²H₄)aniline as a light yellow liquid.

### Step 3: 1,4-diethyl (2E)-2-[[3-chloro(²H₄)phenyl]imino]butanedioate

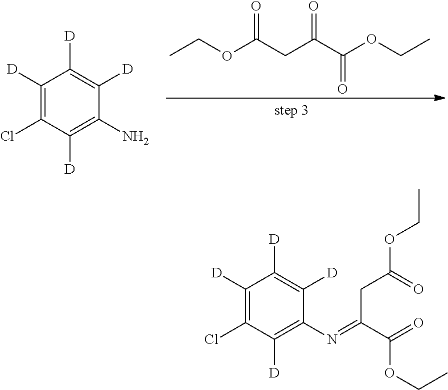

A solution of 3-chloro(²H₄)aniline (4 g, 30.4 mmol, 1.00 equiv) (step 2) and 1,4-diethyl 2-oxobutanedioate (8.6 g, 45.6 mmol, 1.50 equiv) in acetic acid (20 mL) was stirred at 45° C. overnight. The reaction was then quenched by the addition of water/ice. The pH value of the solution was adjusted to 7-8 with sodium hydroxide (35%). The resulting solution was extracted three times with MTBE, the combined organic layer were dried over anhydrous sodium sulfate and concentrated under vacuum. The residue liquid was applied onto a silica gel column with ethyl acetate/petroleum ether (1:9). This resulted in 1.6 g (17%) of 1,4-diethyl (2E)-2-[[3-chloro(²H₄)phenyl]imino]butanedioate as a light yellow liquid.

### Step 4: ethyl 7-chloro-4-oxo-1,4-dihydro(5,6,8-²H₃)quinoline-2-carboxylate

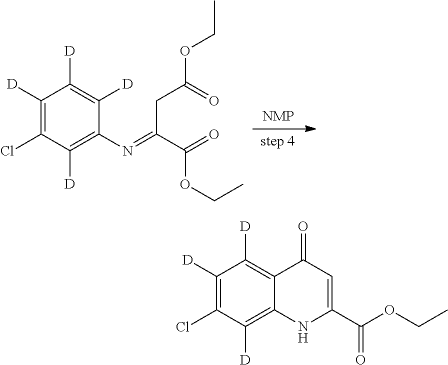

A solution of 1,4-diethyl (2E)-2-[[3-chloro(²H₄)phenyl]imino]butanedioate (1.5 g, 4.97 mmol, 1.00 equiv) (step 3) in NMP (10 mL) was introduced into a 40 mL sealed tube. The reaction mixture was irradiated with microwave radiation for 25 minute at 220° C. The reaction mixture was then cooled to room temperature and quenched by water/ice. The resulting solution was extracted with ethyl acetate (3×20 mL), and the combined organic layer was washed with saturated brine (1×20 mL), dried over anhydrous sodium sulfate, and concentrated under vacuum. The crude product was re-crystallized from EA: N-hexane in the ratio of 1:4. The precipitated solid were filtered off to afford 560 mg (44%) of ethyl 7-chloro-4-oxo-1,4-dihydro(5,6,8-²H₃)quinoline-2-carboxylate as a brown solid.

### Step 5: 7-chloro-4-oxo-1,4-dihydro(5,6,8-²H₃)quinoline-2-carboxylic acid

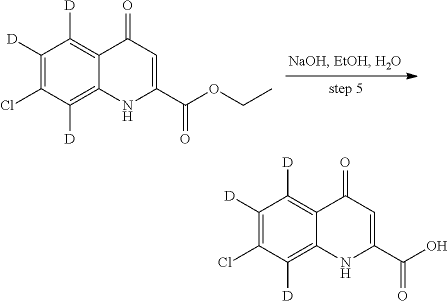 [SMILES: [2H]c1c(Cl)c([2H])c2[nH]c(C(=O)OCC)cc(=O)c2c1[2H] ; [2H]c1c(Cl)c([2H])c2[nH]c(C(=O)O)cc(=O)c2c1[2H]]

To a solution of ethyl 7-chloro-4-oxo-1,4-dihydro(5,6,8-²H₃)quinoline-2-carboxylate (560 mg, 2.20 mmol, 1.00 equiv) (step 4) in ethanol (9 mL) was added a solution of sodium hydroxide (265 mg, 6.62 mmol, 3.00 equiv) in water(3 mL). The resulting solution was stirred for 4 h at room temperature. The resulting mixture was concentrated under vacuum and the residue was dissolved into a small amount of water and the pH value of the solution was adjusted to 4 with hydrogen chloride (1 mol/L). The precipitated solids were collected by filtration and oven dried to afford 310 mg of product which was contained another structural isomer (5-isomer) need to be separated. The crude product was purified by Prep-HPLC with the following conditions: Column: XBridge Prep C18 OBD Column, 5 um, 19*150 mm; Mobile Phase A: Waters (0.05% NH3H2O), Mobile Phase B: ACN; Flow rate: 20 mL/min; Gradient: 2% B to 12% B in 10 min; 254/220 nm. This resulted in 108 mg (22%) of 7-chloro-4-oxo-1,4-dihydro(5,6,8-²H₃)quinoline-2-carboxylic acid as a white solid. ¹H NMR (400 MHz, Methanol-d₄) δ: 6.96 (s, 1H).

The following compounds can generally be made using the methods described above:

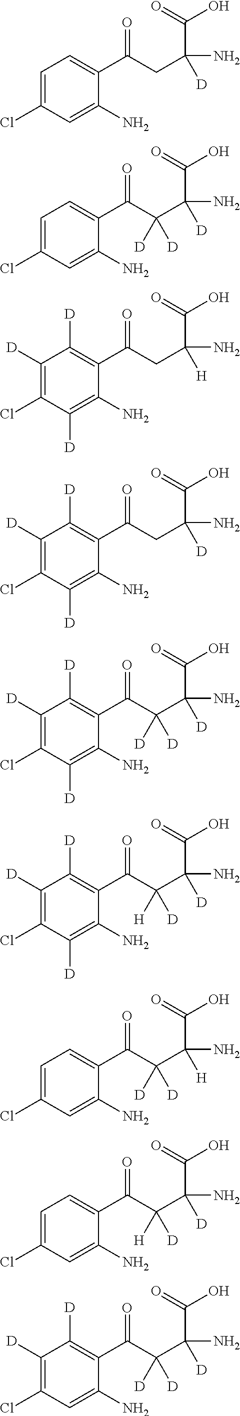

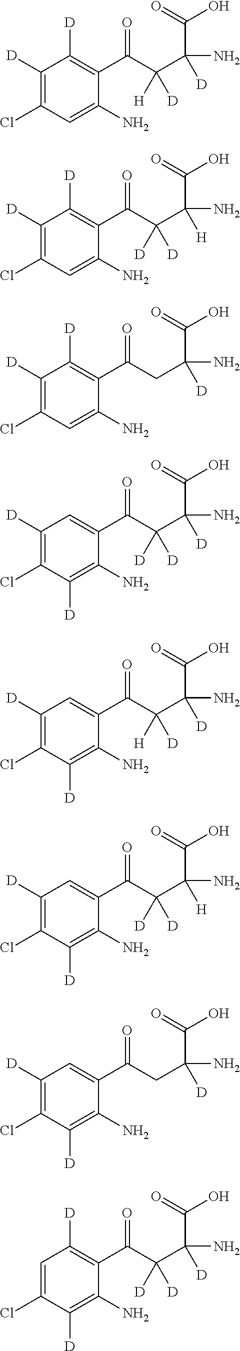

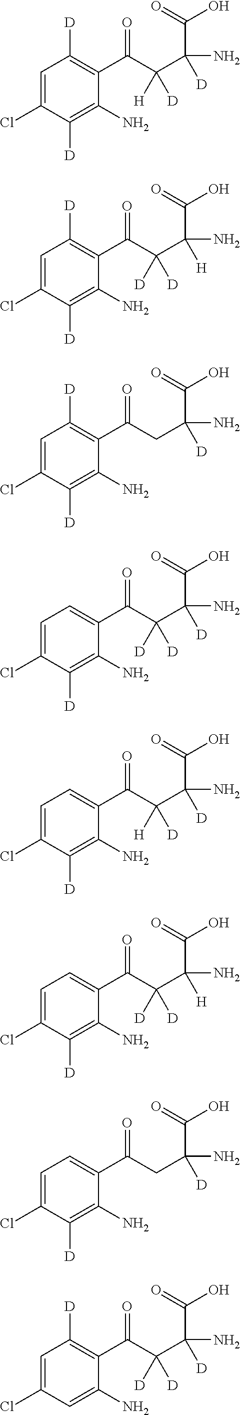

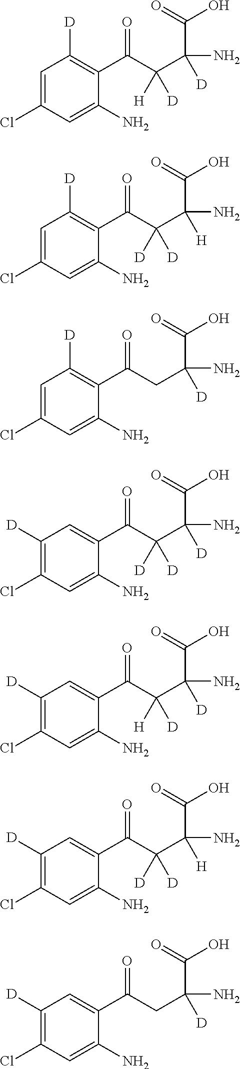

All enantiomers and diastereomers of the foregoing compounds are contemplated herein. Both (R) and (S) racemates of the compounds are contemplated. In certain embodiments, each of the compounds disclosed herein will have (S) stereochemistry at the α-carbon. For example, such compounds might have a formula chosen from:

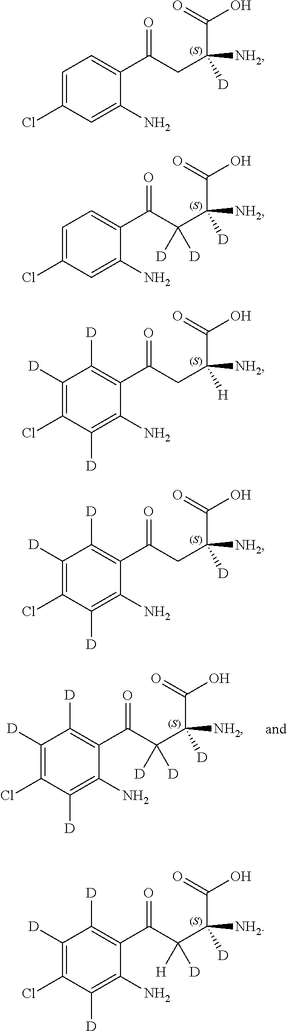

In addition, compounds with one hydrogen and one deuterium at the β-carbon will additionally have an additional stereocenter, and all combinations of enantiomers and diastereomers of such compounds are contemplated herein. For example, for the last compound in the group above, the following stereoisomers are included:

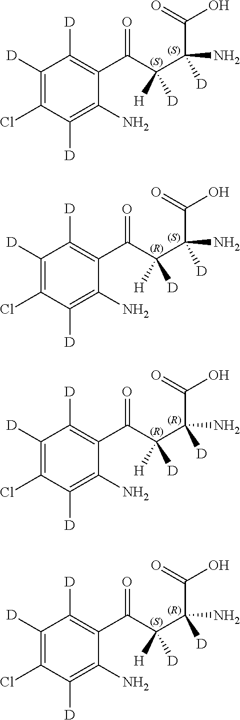

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

In Vitro Liver Microsomal Stability Assay

Human liver microsomal stability assays were conducted at 2 mg per mL liver microsome protein with an NADPH-generating system consisting of NADP (1 mM, pH 7.4), glucose-5-phosphate (5 mM, pH 7.4), and glucose-6-phosphate dehydrogenase (1 unit/mL).

Test compounds were prepared as solutions in DMSO and added to the assay mixture (1 μM, final concentration in incubation) to be incubated at 37±1° C. Reactions were initiated with the addition of cofactor and were stopped at 0, 60, 120, or 240 min after cofactor addition with stop reagent (0.2 mL acetonitrile). Samples were centrifuged (920×g for 10 min at 10° C.) in 96-well plates. Supernatant fractions were analyzed by LC-MS/MS to determine the percent remaining and estimate the degradation half-life of the test compounds.

In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

Monoamine Oxidase A Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

Monooamine Oxidase B Inhibition and Oxidative Turnover

The procedure is carried out as described in Uebelhack, Pharmacopsychiatry 1998, 31(5), 187-192, which is hereby incorporated by reference in its entirety.

### Kynurenine Aminotransferase Assay

The inhibition of kynurenine aminotransferases may be assessed by methods known in the art. See, e.g., Wong J et al., Development of a microplate fluorescence assay for kynurenine aminotransferase, Anal Biochem. 2011 Feb. 15, 409(2):183-8 (epub 2010 Nov. 6) (describing an assay for KATI which could be adapted for other KATs) and Passera E et al., Human kynurenine aminotransferase II—reactivity with substrates and inhibitors, FEBS J. 2011 June; 278(11):1882-900 (epub 2011 Apr. 28).

### Inhibition of [3H]TCP Binding to the Rat NMDA Receptor

The procedure is carried out as described in Goldman et al, FEBS Letters 1985, 190(2), 333-336.

### Rat Model for Hypoxia-Induced Neurodegeneration and NMDA-Antagonist Neuroprotection

The procedure is carried out as described in Reeker et al, Canadian Journal of Anaesthesia 2000, 37(6), 572-578.

### Forced Swim Test

Compounds with antidepressant activity reduce the time of mouse immobility as measured by the “forced swim test” described by Trullos et al., “Functional antagonists at the NMDA receptor complex exhibit antidepressant actions,” Eur. J. Pharm., 185:1-10 (1990) and references therein. Mice are placed individually in a cylinder (i.e., having a diameter of 10 cm and height of 25 cm) filled with water (6 cm) at 22-25° C. The duration of immobility is scored during the last four minutes of a six minute test. The compounds are expected to be active in this test.

### Elevated Plus Maze

Compounds with antidepressant activity increase both the percentage of time and percentage of entries into the open arms of an elevated plus-maze as described by Trullos et al., “L-Aminocyclopropanecarboxylates exhibit antidepressant and anxiolytic actions in animal models,” Eur. J. Pharm., 203:379-385 (1991). A mouse is placed at the intersection of the maze arms so that its head is in the center of the platform. The mouse is then scored as being in the open or enclosed arms. Arm entries are recorded and the percentage of time in each aim, as well as the percentage of entries, are calculated. The compounds are expected to be active in this test.

### NMDA Induced Seizures

Compounds which have anticonvulsant activity for convulsions involving the NMDA receptor are active in a test described by Koek et al., Mechanisms for Neuromodulation and Neuroprotection. pp 665-671, Kamenka et al., eds., NPP Books, Ann Arbor, Mich., 1992. Test compounds are injected into mice at 15 minutes or 30 minutes before an ip injection of NMDA, icy or ip, respectively. ED₅₀ is determined by comparing the percentage of mice that die after 30 minutes to a group of mice that receive NMDA alone. The compounds are expected to be active in this test.

From the foregoing description, one skilled in the art can easily ascertain the essential characteristics of this invention, and without departing from the spirit and scope thereof, can make various changes and modifications of the invention to adapt it to various usages and conditions.

1. A compound of Formula I: 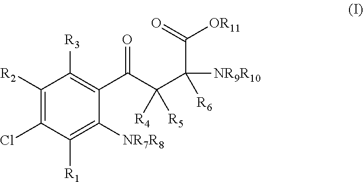
or a salt thereof, wherein:
R₁-R₁₁ are, independently, hydrogen or deuterium; and
at least one of R₁-R₁₁ is deuterium.

2. The compound of claim 1, having Formula II: 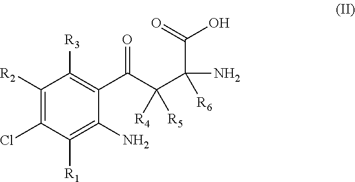
or a salt thereof, wherein:
R₁-R₆ are, independently, hydrogen or deuterium; and
at least one of R₁-R₆ is deuterium.

3. The compound of claim 2, wherein at least one of R₁-R₆ independently has deuterium enrichment of no less than about 10%.

4. The compound of claim 2, wherein at least one of R₁-R₆ independently has deuterium enrichment of no less than about 50%.

5. The compound of claim 2, wherein at least one of R₁-R₆ independently has deuterium enrichment of no less than about 90%.

6. The compound of claim 2, wherein at least one of R₁-R₆ independently has deuterium enrichment of no less than about 98%.

7. The compound of claim 2, wherein said compound is: 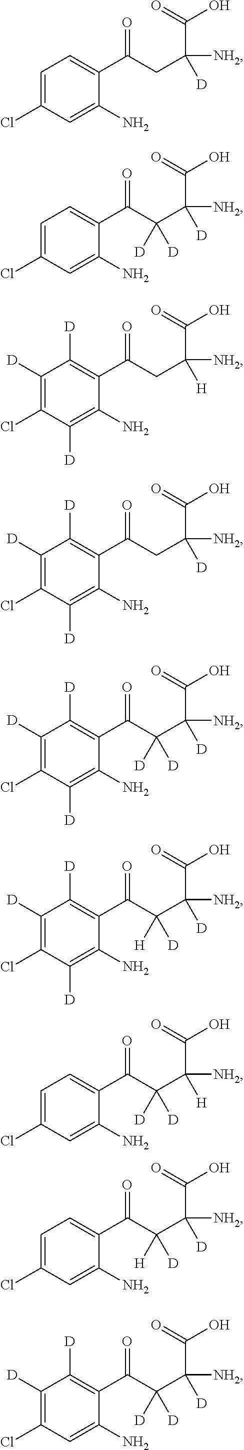 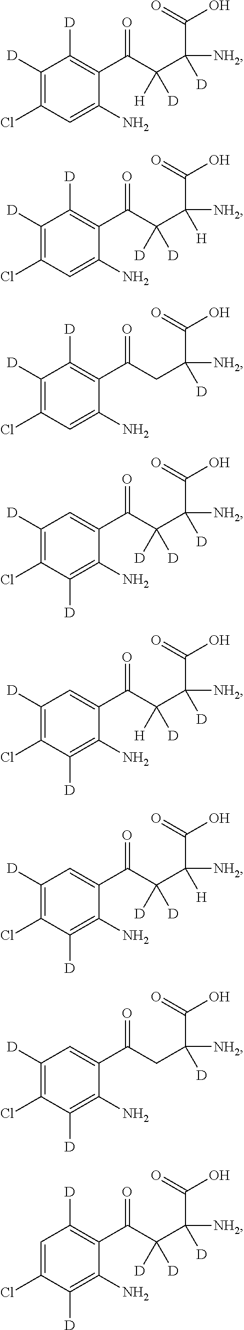 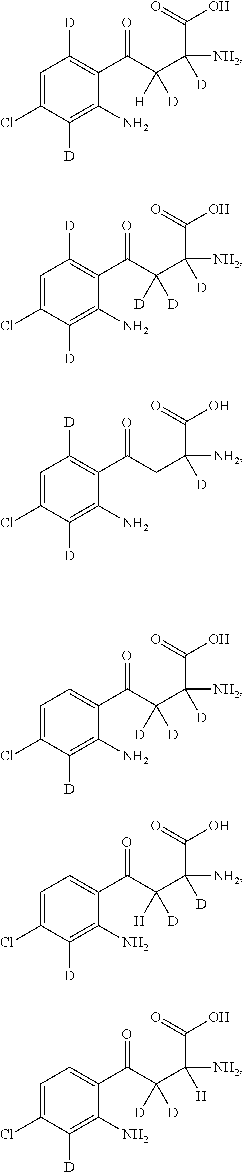 [SMILES: [2H]c1cc(Cl)c([2H])c(N)c1C(=O)C([2H])C([2H])(N)C(=O)O ; [2H]c1cc(Cl)c([2H])c(N)c1C(=O)C([2H])([2H])C([2H])(N)C(=O)O ; [2H]c1cc(Cl)c([2H])c(N)c1C(=O)CC([2H])(N)C(=O)O ; [2H]c1c(Cl)ccc(C(=O)C([2H])([2H])C([2H])(N)C(=O)O)c1N ; [2H]c1c(Cl)ccc(C(=O)C([2H])C([2H])(N)C(=O)O)c1N ; [2H]c1c(Cl)ccc(C(=O)C([2H])([2H])C(N)C(=O)O)c1N] 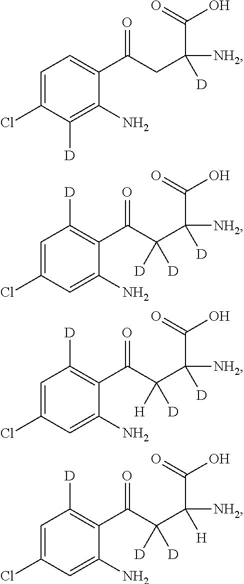 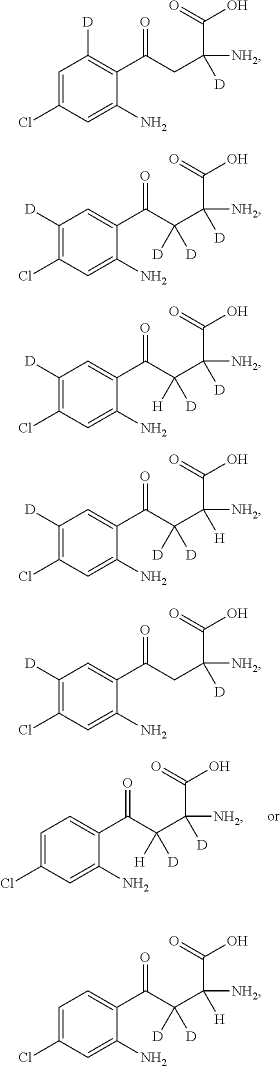
or a salt thereof.

8. The compound of claim 2, which is: 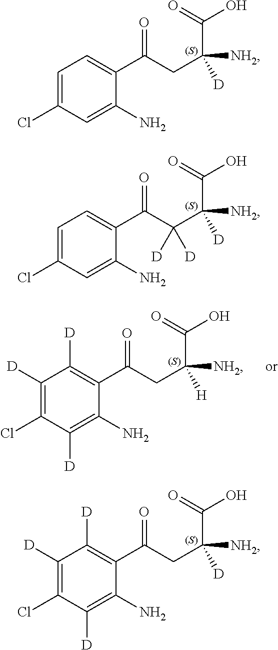
or a salt thereof.

9. The compound of claim 8, wherein each position represented as D has deuterium enrichment of no less than about 10%.

10. The compound of claim 8, wherein each position represented as D has deuterium enrichment of no less than about 50%.

11. The compound of claim 8, wherein each position represented as D has deuterium enrichment of no less than about 90%.

12. The compound of claim 8, wherein each position represented as D has deuterium enrichment of no less than about 98%.

13. The compound of claim 8, which is 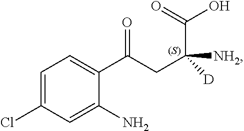
or a salt thereof.

14. The compound of claim 8, which is 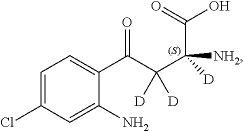
or a salt thereof.

15. The compound of claim 8, which is 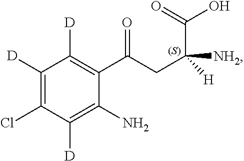
or a salt thereof.

16. The compound of claim 8, which is 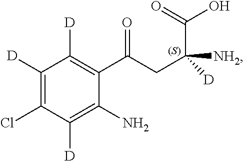
or a salt thereof.

17-20. (canceled)

21. A pharmaceutical composition comprising the compound of claim 1 and a pharmaceutically acceptable carrier.

22. A method of treating a NMDA receptor-mediated disorder comprising administering to a patient in need thereof a therapeutically effective amount of the compound of claim 1.

23. A method of treating a neuropsychiatric disorder, a neurodegenerative disorder, a seizure disorder, an age-related cognitive disorder, a perinatal brain disorder, or a disorder of movement involving chorea, dyskinesia, or one or more tics in a patient, comprising administering to the patient a therapeutically effective amount of the compound of claim 1.

24. A method of treating a disorder that is Alzheimer's disease, vascular dementia, Parkinson's disease, Huntington's disease, amyotriphic lateral sclerosis, multiple sclerosis, traumatic brain injury, major depressive disorder, biopolar disorder, schizophrenia, epilepsy, hyperalgesia, neuropathic pain, migraine, Huntington's disease, tardive dyskinesia, Tourette's Syndrome, or L-DOPA associated dyskinesia in a patient, comprising administering to the patient a therapeutically effective amount of the compound of claim 1.

25. The method of claim 24, wherein the disorder is major depressive disorder.

26. A method of enhancing learning, memory, or cognition in a patient, comprising administering to the patient a therapeutically effective amount of the compound of claim 1.

27. The method of claim 22, further comprising administering an additional therapeutic agent.

28. The method of claim 22, further resulting in at least one effect that is:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; or
e. an improved clinical effect during the treatment in said subject per dosage unit thereof as compared to the non-isotopically enriched compound.

29. (canceled)

30. The method of claim 22, wherein the method effects a decreased metabolism of the compound per dosage unit thereof by at least one polymorphically-expressed metabolizing enzyme isoform in the subject, as compared to the corresponding non-isotopically enriched compound.

31. (canceled)

32. A method of increasing exposure of 7-chlorokynurenic acid in the brain of a subject, comprising administering to the subject an amount of the compound of claim 1, wherein the amount is effective to increase exposure of 7-chlorokynurenic acid in the brain.

33-38. (canceled)
